(19) United States
(12) Patent Application Publication
(10) Pub. No.: US 2005/0089498 A1
(43) Pub. Date: Apr. 28, 2005

(54) Cosmetic compositions containing first and second film forming polymers

(76) Inventors: Anjali Abhimanyu Patil, Westfield, NJ (US); Joseph Frank Calello, Bridgewater, NJ (US); Frank Charles Pagano, Monroe Township, NJ (US); Robert Walter Sandewicz, Monroe Township, NJ (US)

Correspondence Address: Revlon Consumer Products Corporation;Law Department, 237 Park Avenue, New York, NY, 10017, US

(21) Appl. No.: 10/692,663

(22) Filed: Oct. 24, 2003

(51) Int. Cl. A61K 7/021; A61K 7/06; A61K 7/11

(52) U.S. Cl. 424/70.122

(57) ABSTRACT

A cosmetic composition comprising a first film forming siloxane polymer, and a second film forming polymer obtained by polymerizing siloxane monomers and at least one monomer selected from the group consisting of ethylenically unsaturated monomers, urethanes, amides, and mixtures thereof, said polymers solvated or dispersed in a cosmetically acceptable nonpolar oil.

### TECHNICAL FIELD

The invention is in the field of cosmetic compositions for application to keratinous surfaces such as skin, hair, or nails for the purpose of coloring, conditioning, or beautifying the keratinous surface.

### BACKGROUND OF THE INVENTION

Manufacturers of cosmetic products are on an eternal quest to formulate cosmetic compositions that provide better films on keratinous surfaces. The ideal cosmetic film lasts until the consumer wants to remove it by washing with water or using remover compositions. At the same time the film provides a very natural, aesthetic appearance on the keratinous surface without looking fake or “made up”. A suitable cosmetic film should permit the underlying keratinous surface to breathe, seal in moisture, and exhibit a superficially attractive appearance that is not too matte.

Most often, polymers are incorporated into cosmetic compositions to form the cosmetic film. Generally, such polymers contain many repeating units, or monomers, that give the polymer substantive, film forming properties. Such polymers may be natural or synthetic. Natural polymers such as cellulosics, gums, and resins, have been used as film formers in cosmetics for many years. In more recent years, as polymer chemistry has advanced, polymer manufacturers have been able to manufacture a wide variety of synthetic polymers for use in cosmetics. In general, synthetic polymers fall into one of two classes: silicone polymers (based upon silicon and oxygen), or organic polymers comprised of one or more ethylenically unsaturated monomers (such as acrylates, ethylene, amides, etc). Certain synthetic polymers that contain both siloxane monomers and ethylenically unsaturated monomers are also known.

While organic synthetic polymers comprised of ethylenically unsaturated monomers are excellent film formers, they sometimes do not exhibit optimal properties on keratinous surfaces such as skin. Skin is a very dynamic substrate that is in constant movement so cosmetic films that are affixed to skin or lips must exhibit some degree of plasticity. Synthetic organic polymers do not always exhibit the necessary plasticity, and will sometimes crack on dynamic keratinous surfaces such as skin. For this reason, synthetic organic polymers are not as widely used in cosmetic compositions that are applied to skin.

On the other hand, silicone polymers are excellent film formers and have been used to form cosmetic films in many successful commercial products. While silicones provide excellent wear and adhesion in general, organic synthetic polymers often exhibit better wear and adhesion. Silicone acrylate copolymers were fashioned to create polymers that have the best features of both polymer classes. Silicone acrylate copolymers have the excellent wear properties of acrylics, and because they are partly silicone in composition they tend not to exhibit the brittleness sometimes associated with organic synthetic polymers.

It has been found that the combination of a silicone film forming polymer with a second polymer comprised of a mixture of silicone monomers and organic monomers, in particular ethylenically unsaturated monomers, dispersed or solvated in a cosmetically acceptable carrier containing at least one nonpolar oil, provides a cosmetic composition with excellent wear, adhesion, and, in some cases, shine.

It is an object of the invention to provide a cosmetic composition with excellent wear and adhesion to keratinous surfaces.

It is another object of the invention to provide a cosmetic composition that provides a glossy finish to the surface to which it is applied.

It is another object of the invention to provide a lipstick composition that is long wearing and provides a glossy finish.

### SUMMARY OF THE INVENTION

The invention comprises a cosmetic composition comprising a first film forming siloxane polymer, and a second film forming polymer comprised of siloxane monomers and a monomer selected from the group consisting of ethylenically unsaturated monomers, urethanes, amides, and mixtures thereof; both of said polymers solvated or dispersed in a cosmetically acceptable carrier containing at least one nonpolar oil.

The invention further comprises a method for improving the properties of a cosmetic film forming composition on a keratinous surface by including in said composition the combination of a first siloxane film forming polymer and a second film forming polymer comprised of siloxane monomers and a second monomer selected from the group consisting of ethylenically unsaturated monomers, urethanes, amides, and mixtures thereof.

### DETAILED DESCRIPTION

All percentages mentioned herein are percentages by weight unless otherwise noted.

A. The First Film Forming Polymer

The composition of the invention comprises a first film forming polymer which is a silicone or siloxane polymer. The term “silicone polymer” or “siloxane polymer” means a polymer comprised of a backbone of repeating silicon and oxygen atoms, and which comprises one or more of monofunctional, difunctional, trifunctional or tetrafunctional siloxy units. The first film forming polymer may be present in an amount ranging from about 0.001-80%, preferably about 0.01-75%, more preferably about 1-40% by weight of the total composition.

The term monofunctional means that the unit contains one oxygen atom which is shared by 2 silicon atoms when the monofunctional unit is polymerized with one or more of the other types of units. In silicone nomenclature used by those skilled in the art, a monofunctional siloxy unit is designated by the letter “M”, and means a unit having the general formula:
R₁R₂R₃SiO_{1/2}
wherein R₁, R₂, and R₃ are each independently C₁₋₃₀, preferably C₁₋₁₀, more preferably C₁₋₄ straight or branched chain alkyl, which may be substituted with phenyl or one or more hydroxyl groups; phenyl; or hydrogen. The SiO_{1/2} designation means that the oxygen atom in the monofunctional unit is bonded to, or shared, with another silicon atom when the monofunctional unit is polymerized with one or more of the other types of units. For example, when R₁, R₂, and R₃ are methyl the resulting monofunctional unit is of the formula: 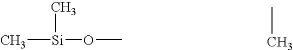
When this monofunctional unit is polymerized with one or more of the other units the oxygen atom will be shared by another silicon atom, i.e. the silicon atom in the monofunctional unit is bonded to ½ of this oxygen atom.

The term “difunctional siloxy unit” is generally designated by the letter “D” in standard silicone nomenclature. If the D unit is substituted with substituents other than methyl the “D′” desigation is sometimes used, which indicates a substituent other than methyl. For purposes of this disclosure, a “D” unit has the general formula:
R₁R₂SiO_{2/2}
wherein R₁ and R₂ are defined as above. The SiO_{2/2} designation means that the silicon atom in the difunctional unit is bonded to two oxygen atoms when the unit is copolymerized with one or more of the other units. For example, when R₁, R₂, are methyl the resulting difunctional unit is of the formula: 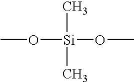
When this difunctional unit is polymerized with one or more of the other units the silicon atom will be bonded to two oxygen atoms, i.e. will share two halves of an oxygen atom.

The term “trifunctional siloxy unit” is generally designated by the letter “T” in standard silicone nomenclature. A “T” unit has the general formula:
R₁SiO_{3/2}
wherein R₁ is as defined above. The SiO_{3/2} designation means that the silicon atom is bonded to three oxygen atoms when the unit is copolymerized with one or more of the other units. For example when R₁ is methyl the resulting trifunctional unit is of the formula: 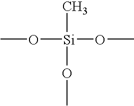
When this trifunctional unit is polymerized with one or more of the other units, the silicon atom shares three oxygen atoms with other silicon atoms, i.e. will share three halves of an oxygen atom.

The term “tetrafunctional siloxy unit” is generally designated by the letter “Q” in standard silicone nomenclature. A “Q” unit has the general formula:
SiO_{4/2}
The SiO_{4/2} designation means that the silicon shares four oxygen atoms (i.e. four halves) with other silicon atoms when the tetrafunctional unit is polymerized with one or more of the other units. The SiO_{4/2} unit is best depicted as follows:

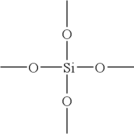

The film forming silicone polymer used in the compositions of the invention may be any combination of M, D, T, or Q units so long as the polymer is capable of forming a cosmetic film on a keratinous surface that includes skin, hair, or nails.

The film forming silicone polymer may be a liquid, semi-solid, or solid at room temperature. It may be a gum or resin. The term “gum” generally means a high molecular weight silicone polymer that may be cross-linked or uncrosslinked, and preferably comprises M and D units, and possibly some T or Q units. Examples of suitable silicone gums include high molecular weight polydimethylsiloxanes having viscosities in the range of about 100,000 to 10,000,000, preferably about 500,000 to 8,000,000 centipoise at 25° C. In the case where the silicone gum is crosslinked, one or more of the M, D, or T units may be substituted with hydrogen atoms, which will react with suitable cross linking groups such as vinyl or alpha omega dienes, or possibly other reactive sites on substituted M, D, or T units. For example, cross-linking can occur when a polydimethyl-methylhydrogen siloxane is polymerized in the usual manner with a second polydimethylsiloxane that is substituted with one or more vinyl or alpha omega diene functional groups.

The silicone film forming polymer used in the compositions of the invention is preferably a silicone resin. The term “silicone resin” when used herein means a silicone containing T, MT, or MQ units. The term “T” means that the silicone comprises mostly T units, as above defined, either alone or in combination with D or M units. The term “MT” means that the silicone contains at least M and T units as defined above and possibly minor amounts of other types of units. The term “MQ” means that the silicone resin comprises at least M and Q units as defined above and possibly minor amounts of other types of units.

Typically T or MT silicones are referred to as silsesquioxanes, and in the case where M units are present methylsilsesquioxanes. Preferred are T silicones having the following general formula:
(R₁ SiO_{3/2})ₓ where x ranges from about 1 to 100,000.
wherein R₁ is as defined above. In another embodiment the preferred silicone resin is an MT resin referred to as polymethylsilsesquioxane which are silsesquioxanes containing methyl groups. Particularly preferred are polymethylsilsesquioxanes manufactured by Wacker Chemie under the Resin MK designation. This polymethylsilsesquioxane is a polymer comprise of T units and, optionally one or more D (preferably dimethylsiloxy) units. This particularly polymer may have ends capped with ethoxy groups, and/or hydroxyl groups, which may be due to how the polymers are made, e.g. condensation in aqueous or alcoholic media. Other suitable polymethylsilsesquioxanes that may be used as the film forming polymer include those manufactured by Shin-Etsu Silicones and include the “KR” series, e.g. KR-220L, 242A, and so on. These particular silicone resins may contain endcap units that are hydroxyl or alkoxy groups which may be present due to the manner in which such resins are manufactured.

The silicone resin may also be an MQ resin, also generally referred to as a siloxy silicate resin. Such silicones generally have the formula:
[R₁R₂R₃SiO_{1/2}]ₓ[SiO_{4/2}]_{y}
wherein R₁, R₂, and R₃ are each independently as defined above, and, preferably, x and y are such that the ratio of [R₁R₂R₃SiO_{1/2}] to [SiO_{4/2}] units is 0.5 to 1 to 1.5 to 1. Preferably R₁, R₂, and R₃ are a C₁₋₆ alkyl, and more preferably are methyl and x and y are such that the ratio of [R₁R₂R₃SiO_{1/2}] to [SiO_{4/2}] units is 0.75 to 1. Most preferred is this trimethylsiloxy silicate containing 2.4 to 2.9 weight percent hydroxyl groups which is formed by the reaction of the sodium salt of silicic acid, chlorotrimethylsilane, and isopropyl alcohol. The manufacture of trimethylsiloxy silicate is set forth in U.S. Pat. Nos. 2,676,182; 3,541,205; and 3,836,437, all of which-are hereby incorporated by reference. Trimethylsiloxy silicate as described is available from Dow Corning Corporation under the tradename 749 Fluid, which is a blend of about 40-60% volatile silicone and 40-60% trimethylsiloxy silicate. Dow Corning 2-0749 in particular, is a fluid containing about 50% trimethylsiloxy silicate and about 50% cyclomethicone. The fluid has a viscosity of 200-700 centipoise at 25.degree. C., a specific gravity of 1.00 to 1.10 at 25.degree. C., and a refractive index of 1.40-1.41. Also suitable are MQ resins manufactured by GE Silicones under the tradename SR1000, and Wacker silicones under the tradename TMS 803.

The film forming silicone polymers that may be used in the composition are made according to processes well known in the art. In general siloxane polymers are obtained by hydrolysis of silane monomers, preferably chlorosilane monomers. The chlorosilanes are hydrolyzed to silanols and then condensed to form siloxanes. The hydrolysis and condensation may leave some residual hydroxy or alkoxy functionality on the siloxane. The silicone film forming polymers used in the compositions of the invention are generally made in accordance with the methods set forth in Silicon Compounds (Silicones), Bruce B. Hardman, Arnold Torkelson, General Electric Company, Kirk-Othmer Encyclopedia of Chemical Technology, Volume 20, Third Edition, pages 922-962, 1982, which is hereby incorporated by reference in its entirety.

Preferred compositions according to the invention contain one or more of a T, MT, or MQ silicone polymer. Most preferred is a T or MQ silicone polymer or mixtures thereof.

B. The Second Film Forming Polymer

The second film forming polymer is a polymer obtained by polymerization of siloxane monomers and ethylenically unsaturated monomers. Preferably, the second film forming polymer is present at amounts ranging from about 0.001-80%, preferably about 0.01-75%, more preferably about 0.1-65% by weight of the total composition.

The siloxane monomers in the second film forming polymer may be obtained by polymerization of any one or more of the M, D, T, or Q units as set forth above with any one or more ethylenically unsaturated monomers.

The ethylenically unsaturated monomers may include repeating C₁₋₃₀ alkylenes such as ethylene, propylene, butylene, and the like; as well as acrylic acid, methacrylic acid, either alone or esterified with C₁₋₃₀ alkanols; styrene, vinyl pyrrolidone, amides, urethanes, and the like.

One type of ethylenically unsaturated monomer is of the formula: 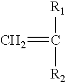
wherein R₁, and R₂ are each independently H, halogen, hydroxyl, fluoroalkyl, a C₁₋₃₀ straight or branched chain alkyl, aryl, aralkyl; R₂ is a pyrrolidone, or a substituted or unsubstituted aromatic, alicyclic, or bicyclic ring where the substitutents are C₁₋₃₀ straight or branched chain alkyl, or COOM or OCOM herein M is a C₁₋₃₀ straight or branched chain alkyl, pyrrolidone, or a substituted or unsubstituted aromatic, alicylic, or bicyclic ring where the substitutents are C₁₋₃₀ straight or branched chain alkyl.

Another type of monomer that may be polymerized with the siloxane monomers is a urethane monomer. Urethanes are generally formed by the reaction of polyhydroxy compounds with diisocyanates, as follows:

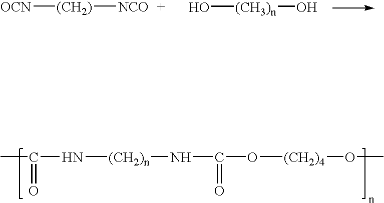

Another type of monomer that may be polymerized with the siloxane monomers comprises amide groups, preferably having the the following formula: 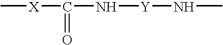
wherein X and Y are each independently linear or branched alkylene having ₁₋₄₀ carbon atoms, which may be substituted with one or more amide, hydrogen, alkyl, aryl, or halogen substituents. Particularly preferred are silicone polyamides as set forth in U.S. Pat. No. 6,353,076, which is hereby incorporated by reference in its entirety.

Preferred are polymers of siloxane monomers and esters of methacrylic or acrylic acid, more generally referred to as silicone acrylate copolymers.

The resulting copolymers may be random, radial, brached, or graft or block copolymers. The term “graft copolymer” is familiar to one of ordinary skill in polymer science and is used herein to describe the copolymers which result by adding or “grafting” polymeric side chain moieties (i.e. “grafts”) onto another polymeric moiety referred to as the “backbone”. The backbone may have a higher molecular weight than the grafts. Thus, graft copolymers can be described as polymers having pendant polymeric side chains, and which are formed from the “grafting” or incorporation of polymeric side chains onto or into a polymer backbone. The polymer backbone can be a homopolymer or a copolymer. The graft copolymers are derived from a variety of monomer units. The term “block copolymer” means that the monomeric portions of the polymer exist in blocks, for example blocks of one monomer and blocks of another monomer which, in turn, may lead to a polymer with hard and soft segments. The term “random” means that the different monomers in the polymer are distributed throughout the polymer in random configuration.

One type of silicone acrylate copolymer that may be used as the film forming polymer is a vinyl-silicone graft or block copolymer having the formula: 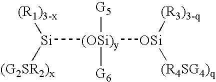
wherein G₅ represents monovalent moieties which can independently be the same or different selected from the group consisting of alkyl, aryl, aralkyl, alkoxy, alkylamino, fluoroalkyl, hydrogen, and -ZSA; A represents a vinyl polymeric segment consisting essentially of a polymerized free radically polymerizable monomer, and Z is a divalent linking group such as C₁₋₁₀ alkylene, aralkylene, arylene, and alkoxylalkylene, most preferably Z methylene or propylene.

G₆ is a monovalent moiety which can independently be the same or different selected from the group consisting of alkyl, aryl, aralkyl, alkoxy, alkylamino, fluoroalkyl, hydrogen, and -ZSA;
G₂ comprises A;
G₄ comprises A;
R₁ is a monovalent moiety which can independently be the same or different and is selected from the group consisting of alkyl, aryl, aralkyl, alkoxy, alkylamino, fluoroalkyl, hydrogen, and hydroxyl; but preferably C₁₋₄ alkyl or hydroxyl, and most preferably methyl.
R₂ is independently the same or different and is a divalent linking group such as C₁₋₁₀ alkylene, arylene, aralkylene, and alkoxyalkylene, preferably C₁₋₃ alkylene or C₇₋₁₀ aralkylene, and most preferably —CH₂— or 1,3-propylene, and
R₃ is a monovalent moiety which is independently alkyl, aryl, aralkyl, alkoxy, alkylamino, fluoroalkyl, hydrogen, or hydroxyl, preferably C₁₋₄ alkyl or hydroxyl, most preferably methyl;
R₄ is independently the same or different and is a divalent linking group such as C₁₋₁₀ alkylene, arylene, aralkylene, alkoxyalkylene, but preferably C₁₋₃ alkylene and C₇₋₁₀ alkarylene, most preferably —CH₂— or 1,3-propylene;
x is an integer of 0-3;
y is an integer of 5 or greater; preferably 10 to 270, and more preferably 40-270; and
q is an integer of 0-3.

These polymers are described in U.S. Pat. No. 5,468,477, which is hereby incorporated by reference. One type of such polymer is poly(dimethylsiloxane)-g-poly(isobutyl methacrylate), which is manufactured by 3-M Company under the tradename 3M Silicone Plus Polymer VS 70. This polymer may be purchased in the dry particulate form, or as a solution where the polymer is dissolved in one or more volatile solvents such as isododecane. Preferred is where the polymer is in dry particulate form, and as such it can be dissolved in one or more of the volatile solvents found in the gel composition. This polymer has the CTFA name Polysilicone-6.

Another type of such a polymer comprises a vinyl, methacrylic, or acrylic backbone with pendant siloxane groups and pendant fluorochemical groups. Such polymers preferably comprise comprise repeating A, C, D and optionally B monomers wherein:
A is at least one free radically polymerizable acrylic or methacrylic ester of a 1,1,-dihydroperfluoroalkanol or analog thereof, omega-hydridofluoroalkanols, fluoroalkylsulfonamido alcohols, cyclic fluoroalkyl alcohols, and fluoroether alcohols,
B is at least one reinforcing monomer copolymerizable with A,
C is a monomer having the general formula X(Y)nSi(R)3-m Z.m wherein
X is a vinyl group copolymerizable with the A and B monomers,
Y is a divalent linking group which is alkylene, arylene, alkarylene, and aralkylene of 1 to 30 carbon atoms which may incorporate ester, amide, urethane, or urea groups,
n is zero or 1;
m is an integer of from 1 to 3,
R is hydrogen, C₁₋₄ alkyl, aryl, or alkoxy,
Z is a monovalent siloxane polymeric moiety; and
D is at least one free radically polymerizable acrylate or methacrylate copolymer.

Such polymers and their manufacture are disclosed in U.S. Pat. Nos. 5,209,924 and 4,972,037, which are hereby incorporated by reference. More specifically, the preferred polymer is a combination of A, C, and D monomers wherein A is a polymerizable acrylic or methacrylic ester of a fluoroalkylsulfonamido alcohol, and where D is a methacrylic acid ester of a C₁₋₂ straight or branched chain alcohol, and C is as defined above. An example is a polymer having the general formula: 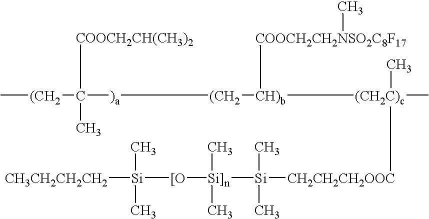
wherein each of a, b, and c has a value in the range of 1-100,000, and the terminal groups are selected from the group consisting of a C₁₋₂₀ straight or branched chain alkyl, aryl, and alkoxy and the like. These polymers may be purchased from Minnesota Mining and Manufacturing Company under the tradenames “Silicone Plus” polymers. Most preferred is poly(isobutyl methacrylate-co-methyl FOSEA)-g-poly(dimethylsiloxane) which is sold under the tradename SA 70-5 IBMMF having the C.T.F.A. name Polysilicone 7.

Another suitable silicone acrylate copolymer is a polymer having a vinyl, methacrylic, or acrylic polymeric backbone with pendant siloxane groups. Such polymers as disclosed in U.S. Pat. Nos. 4,693,935, 4,981,903, 4,981,902, and which are hereby incorporated by reference. Preferably, these polymers are comprised of A, C, and optionally B monomers wherein:
A is at least on free radically polymerizable vinyl, methacrylate, or acrylate monomer;
B, when present, is at least one reinforcing monomer copolymerizable with A,
C is a monomer having the general formula:
   X(Y)ₙSi(R)₃₋ₘZₘ
   wherein:
X is a vinyl group copolymerizable with the A and B monomers;
Y is a divalent linking group;
n is zero or 1;
m is an integer of from 1 to 3;
R is hydrogen, C₁₋₁₀ alkyl, substituted or unsubstituted phenyl, C₁₋₁₀ alkoxy; and
Z is a monovalent siloxane polymeric moiety.

Examples of A monomers are lower to intermediate methacrylic acid esters of C₁₋₁₂ straight or branched chain alcohols, styrene, vinyl esters, vinyl chloride, vinylidene chloride, acryloyl monomers, and so on.

The B monomer, if present, is a polar acrylic or methacrylic monomer having at least one hydroxyl, amino, or ionic group (such as quaternary ammonium, carboxylate salt, sulfonic acid salt, and so on).

The C monomer is as above defined.

Examples of other suitable copolymers that may be used herein, and their method of manufacture, are described in detail in U.S. Pat. No. 4,693,935, Mazurek, U.S. Pat. No. 4,728,571, and Clemens et al., both of which are incorporated herein by reference. Additional grafted polymers are also disclosed in EPO Application 90307528.1, published as EPO Application 0 408 311, U.S. Pat. No. 5,061,481, Suzuki et al., U.S. Pat. No. 5,106,609, Bolich et al., U.S. Pat. No. 5,100,658, Bolich et al., U.S. Pat. No. 5,100,657, Ansher-Jackson, et al., U.S. Pat. No. 5,104,646, Bolich et al., U.S. Pat. No. 5,618,524, issued Apr. 8, 1997, all of which are incorporated by reference herein in their entirety. Also suitable is KP 56 sold by Shin Etsu, which has an INCI name of acrylates/stearyl acrylate/dimethicone acrylates copolymer with a melting point of 25-35° C.; KP 562P, also from Shin Etsu, which is acrylates/behenyl acrylate/dimethicone acrylates copolymer, which has a melting point of 45-55° C.; and KP550 from Shin Etsu having the INCI name of acrylates/dimethicone copolymer.

In the preferred embodiment of the invention, the composition contains an MQ silicone resin in combination with a silicone acrylate copolymer.

C. The Nonpolar Oil

The first and second film forming polymers in the composition are solvated or dispersed in a cosmetically acceptable carrier containing at least one nonpolar oil. The term “oil” means an ingredient that is pourable liquid at room temperature, e.g. 25° C. The viscosity of the oil does not matter so long as it may be poured at ambient temperature.

The term “nonpolar” means that the oil is lipophilic in character, and exhibits a preference for the lipophilic phase of the cosmetic composition. The nonpolar oil is not itself a film forming polymer, but whether in the polymeric form or not, may act as a solvent for one or more of the film forming polymers in the composition, or an emollient or other ingredient that provides beneficial properties. A variety of nonpolar oils are suitable, including silicones, organic oils, esters, paraffinic hydrocarbons, triglycerides, and the like. Such oils are preferably present in amounts ranging from about 0.01-80%, preferably about 0.05-75%, more preferably about 0.1-70% by weight of the total composition. Suitable oils include those set forth below.

The nonpolar oil may be volatile or nonvolatile. The term “volatile” means that the oil has a measureable vapor pressure, or a vapor pressure of at least about 2 mm. of mercury at 20° C. The term “nonvolatile” means that the oil has a vapor pressure of less than about 2 mm. of mercury at 20° C. Suitable volatile oils generally have a viscosity of 0.5 to 10 centistokes at 25° C. Suitable volatile oils include linear silicones, cyclic silicones, paraffinic hydrocarbons, or mixtures thereof.

Cyclic silicones (or cyclomethicones) are of the general formula: 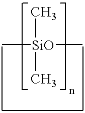
where n=3-6.

Linear volatile silicones in accordance with the invention have the general formula:
(CH₃)₃Si—O—[Si(CH₃)₂—O]ₙ—Si(CH₃)₃
where n=0-7, preferably 0-5.

Linear and cyclic volatile silicones are available from various commercial sources including Dow Corning Corporation and General Electric. The Dow Corning volatile silicones are sold under the tradenames Dow Corning 244, 245, 344, and 200 fluids. These fluids comprise octamethylcyclotetrasiloxane, decamethylcyclopentasiloxane, hexamethyldisiloxane, octamethyltrisiloxane, decamethyltetrasiloxane, and dodecamethylpentasiloxane, and mixtures thereof.

Also suitable as the volatile oils are various straight or branched chain paraffinic hydrocarbons having 5 to 40 carbon atoms, more preferably 8-20 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference. Preferred volatile paraffinic hydrocarbons have a molecular weight of 70-225, preferably 160 to 190 and a boiling point range of 30 to 320, preferably 60-260 degrees C., and a viscosity of less than 10 cs. at 25 degrees C. Such paraffinic hydrocarbons are available from EXXON under the ISOPARS trademark, and from the Permethyl Corporation. Suitable C₁₂ isoparaffins are manufactured by Permethyl Corporation under the tradename Permethyl 99A. Another C₁₂ isoparaffin (isododecane) is distributed by Presperse under the tradename Permethyl 99A. Various C₁₆ isoparaffins commercially available, such as isohexadecane (having the tradename Permethyl R), are also suitable. Transfer resistant cosmetic sticks of the invention will generally comprise a mixture of volatile silicones and volatile paraffinic hydrocarbons.

A wide variety of nonvolatile oils are also suitable for use in the cosmetic compositions of the invention. The nonvolatile oils generally have a viscosity of greater than about 2, preferably about 5 to 10 centipoise at 25° C., and may range in viscosity up to 1,000,000 centipoise at 25° C. Examples of nonvolatile oils suitable for use in the cosmetic compositions of the invention include esters of the formula RCO—OR′ wherein R and R′ are each independently a C₁₋₂₅, preferably a C₄₋₂₀ straight or branched chain alkyl, alkenyl or alkoxycarbonylalkyl or alkylcarbonyloxyalkyl. Examples of such esters include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, neopentylglycol dioctanoate, octyl isononanoate, pentaerythrityl tetraoctanoate, octyl palmitate, dioctyl malate, isononyl isononanoate, tridecyl octanoate, myristyl myristate, octododecanol, and fatty alcohols such as oleyl alcohol, isocetyl alcohol, and the like, as well as the esters disclosed on pages 1670-1676 of the C.T.F.A. Cosmetic Ingredient Dictionary and Handbook, Eighth Edition, 2000, which is hereby incorporated by reference in its entirety.

The oil may also comprise naturally occuring or synthetic carboxylic acid mono-, di, or triglycerides, which may be modified by reaction with alcohols, or other ingredients. In the case where the glyceryl esters are naturally occurring, both vegetable and animal sources may be used. Such glyceryl esters may be formed by the reaction of a C₁₋₄₀ straight or branched chain, saturated or unsaturated carboxylic acids with mono-, di-, or triglycerides. Preferably the carboxylic acid is one or more C₆₋₄₀ fatty acids. Examples of such ingredients include those set forth on pages 1678-1679 under the heading “Fats and Oils” and those set forth on pages 1680-1683 under the heading “Glyceryl Esters and Derivatives (Excluding Fats and Oils) as set forth in the C.T.F.A. Cosmetic Ingredient Dictionary and Handbook, Eighth Edition, 2000, which is hereby incorporated by reference in its entirety. Examples of such oils include, but are not limited to, C12-18 triglycerides, Camellia oils, canola oil, caprylic/capric triglycerides (including lauric, linoleic, myristic, stearic), C 10-18 trigclyerides, emu oil, hydrogenated oils such as castor oil, olive oil, orange oil, Avocado oil, corn oil, coconut oil, cottonseed oil, soybean oil, and mixtures thereof.

Also suitable as the oil are synthetic or semi-synthetic glyceryl esters, e.g. fatty acid mono-, di-, and triglycerides which are natural fats or oils that have been modified. Examples of such ingredients include esters of polyols, or mono-, di-, or trihydric alcohols (such as glycerin, butylene glycol, propylene glycol) and fatty acids (C6-40 carboxylic acids). Examples of such ingredients include polyol monoesters, polyol diesters, polyol triesters, and so on, including for example, acetylated castor oil, glyceryl stearate, diglyceryl diisostearate, polyglyceryl-3-isostearate, polyglyceryl-4-diisostearate, glyceryl dioleate, glyceryl distearate, glyceryl trioctanoate, glyceryl diisotearate, glyceryl linoleate, glyceryl myristate, glyceryl isostearate, PEG castor oils, PEG glyceryl oleates, PEG glyceryl stearates, PEG glyceryl tallowates, fatty esters (such as capric, stearic, isostearic, palmitic, lauric, oleic, ricinoleic, etc.) of polyglycerin having from about 2-20 repeating glycerin units. Examples of such ingredients include polyglyceryl-3 isostearate, polyglyceryl-4-isostearate, polyglyceryl-2-oleate, polyglyceryl-6-ricinoleate, and mixtures thereof.

Also suitable as the oil are nonvolatile hydrocarbons such as isoparaffins, hydrogenated polyisobutene, hydrogenated polydecene, mineral oil, squalene, petrolatum, and so on.

Straight or branched chain fatty alcohols having the formula R—OH, wherein R is a straight or branched chain saturated or unsaturated alkyl having 6-30 carbon atoms, are also suitable oils. Such fatty alcohols include cetyl alcohol, cetearyl alcohol, and the like.

Also suitable as the oil are various lanolin derivatives such as acetylated lanolin, acetylated lanolin alcohol, and so on.

Nonvolatile silicones that are not film forming polymers, both water soluble and water insoluble, are also suitable as the oil component. Such silicones preferably have a viscosity of about 5 to 600,000 centistokes, preferably about 10 to 100,000 centistokes at 25° C. Suitable water insoluble silicones include amodimethicone, bisphenylhexamethicone, dimethicone, hexadecyl methicone, phenyl trimethicone, simethicone, dimethylhydrogensiloxane, stearoxytrimethylsilane, vinyldimethicone, and mixtures thereof.

Water soluble, non-film forming silicones such as dimethicone copolyol, dimethiconol, and the like may be used. Such silicones are available from Dow Corning as the 3225C formulation aid, Dow 190 and 193 fluids, or similar products marketed by Goldschmidt under the ABIL tradename.

Also suitable as the oil are various fluorinated oils such as fluorinated silicones, fluorinated esters, or perfluropolyethers. Particularly suitable are fluorosilicones such as trimethylsilyl endcapped fluorosilicone oil, polytrifluoropropylmethylsiloxanes, and similar silicones such as those disclosed in U.S. Pat. No. 5,118,496 which is hereby incorporated by reference. Perfluoropolyethers like those disclosed in U.S. Pat. Nos. 5,183,589, 4,803,067, 5,183,588 all of which are hereby incorporated by reference, which are commercially available from Montefluos under the trademark Fomblin, are also suitable shine enhancers.

Guerbet esters are also suitable oils. The term “guerbet ester” means an ester which is formed by the reaction of a guerbet alcohol having the general formula: 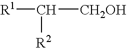
with a carboxylic acid having the general formula:
R³COOH, or
HOOC—R³—COOH
wherein R¹ and R² are each independently a C₄₋₂₀ alkyl and R³ is a substituted or unsubstituted fatty radical such as a C₁₋₅₀ straight or branched chain saturated or unsaturated alkyl or alkylene, or phenyl, wherein the substituents are halogen, hydroxyl, carboxyl, and alkylcarbonylhydroxy. Particularly preferred is a carboxylic acid wherein the R group is such to provide an ingredient known as meadowfoam seed oil.

Preferably, the guerbet ester is a fluoro-guerbet ester which is formed by the reaction of a guerbet alcohol and carboxylic acid (as defined above), and a fluoroalcohol having the following general formula:
CF₃—(CF₂)ₙ—CH₂—CH₂—OH
wherein n is from 3 to 40.

Examples of suitable fluoro guerbet esters are set forth in U.S. Pat. No. 5,488,121 which is hereby incorporated by reference. Suitable fluoro-guerbet esters are also set forth in U.S. Pat. No. 5,312,968 which is hereby incorporated by reference. Most preferred is a guerbet ester having the tentative CTFA name fluoro-octyldodecyl meadowfoamate. This ester is sold by Siltech, Norcross Ga. as Developmental Ester L61125A, under the tradename Silube GME-F.

Preferred is where the nonpolar oil comprises a volatile paraffinic hydrocarbon, more specifically isododecane.

The cosmetic compositions of the invention may be in a variety of forms, including anhydrous and aqueous emulsion. The cosmetic compositions may be in the form of lipstick, blush, concealer, foundation, skin lotions and creams, nail enamel, mascara, eyeshadow, eyeliner, tattoos, and hair care products such as shampoo, conditioner, and the like. If in the emulsion form, the compositions generally comprise about 0.001-90%, preferably about 0.01-80%, more preferably about 0.5-75% water and 0.001-90%, preferably about 0.01-80%, more preferably about 0.5-75% oil.

D. Other Ingredients

The compositions may contain other ingredients such as pigments, particulates, surfactants, waxes, humectants, particulates, pigments, fibers, and the like.

### Waxes

Suitable waxes are animal, vegetable, mineral, and synthetic waxes, or silicone waxes including stearoxydimethicone, stearyl dimethicone, polyethylene, paraffin, ceresin, ozokerite, including but not limited to those set forth in U.S. Pat. No. 5,725,845 which is hereby incorporated by reference in its entirety. Preferred ranges of wax are about 0.5-75%, preferably about 1-65% by weight of the total composition.

### Particulate Matter

In the event the compositions are colored cosmetic compositions, they may contain amounts of particulates ranging from about 0.1-50%, more preferably about 0.5-18% of particulate matter having a particle size of 0.01 to 200, preferably 0.25-100 microns. The particulate matter may be colored or non-colored (for example white) non-pigmentitious powders that may give the cosmetic stick an opaque or semi-opaque quality and contribute to stick structure. Suitable non-pigmentatious powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof. While titanium dioxide is commonly considered to be a white pigment when used in paints, in cosmetic sticks it is used more for its ability to mute color, and/or provide an opaque or semi-opaque finish, then as a colorizing ingredient. The above mentioned powders may be surface treated with lecithin, amino acids, mineral oil, silicone, or various other agents either alone or in combination, which coat the powder surface and render the particles more lipophilic in nature.

The particulate matter component also may comprise various organic and/or inorganic pigments, alone or in admixture with one or more non-pigmentatious powders. The organic pigments are generally various aromatic types including azo, indigoid, triphenylmethane, anthraquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof.

The composition may contain a mixture of both pigmentatious and non-pigmentatious particulate matter. The percentage of pigment used in the particulate matter component will depend on the type of cosmetic being formulated.

### Surfactants

The compositions of the invention may comprise about 0.01-20%, preferably about 0.1-15%, more preferably about 0.5-10% by weight of the total composition of a surfactant. The surfactant may be nonionic, although if the composition is in the form of a shampoo or conditioner it will preferably contain anionic or cationic surfactants, respectively. Suitable nonionic surfactants or emulsifiers include alkoxylated alcohols, or ethers, formed by the reaction of an alcohol with an alkylene oxide, usually ethylene or propylene oxide. Preferably the alcohol is either a fatty alcohol having 6 to 30 carbon atoms. Examples of such ingredients include Beheneth 5-30, which is formed by the reaction of behenyl alcohol and ethylene oxide where the number of repeated ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on. Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol. Preferred are alkoxylated alcohols which are formed by the reaction of stearic acid, methyl glucose, and and ethoxylated alcohol, otherwise known as PEG-20 methyl glucose sesquiisostearate.

Also suitable as the nonionic surfactant are alkyoxylated carboxylic acids, which are formed by the reaction of a carboxylic acid with an alkylene oxide or with a polymeric ether. The resulting products have the general formula: 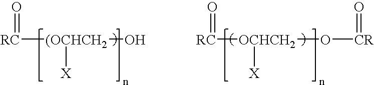
where RCO is the carboxylic ester radical, X is hydrogen or lower alkyl, and n is the number of polymerized alkoxy groups. In the case of the diesters, the two RCO— groups do not need to be identical. Preferably, R is a C₆₋₃₀ straight or branched chain, saturated or unsaturated alkyl, and n is from 1-100.

Also suitable as the nonionic surfactant are monomeric, homopolymeric and block copolymeric ethers. Such ethers are formed by the polymerization of monomeric alkylene oxides, generally ethylene or propylene oxide. Such polymeric ethers have the following general formula: 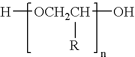
wherein R is H or lower alkyl and n is the number of repeating monomer units, and ranges from 1 to 500.

Other suitable nonionic surfactants include alkoxylated sorbitan and alkoxylated sorbitan derivatives. For example, alkoxylation, in particular, ethoxylation, of sorbitan provides polyalkoxylated sorbitan derivatives. Esterification of polyalkoxylated sorbitan provides sorbitan esters such as the polysorbates. Examples of such ingredients include Polysorbates 20-85, sorbitan oleate, sorbitan palmitate, sorbitan sesquiisostearate, sorbitan stearate, and so on.

Also suitable as nonionic surfactants are silicone surfactants, which are defined as silicone polymers which have at least one hydrophilic radical and at least one lipophilic radical. The silicone surfactant used in the compositions of the invention are organosiloxane polymers that may be a liquid or solid at room temperature. The organosiloxane surfactant is generally a water-in-oil or oil-in-water type surfactant which is, and has an Hydrophile/Lipophile Balance (HLB) of 2 to 18. Preferably the organosiloxane is a nonionic surfactant having an HLB of 2 to 12, preferably 2 to 10, most preferably 4 to 6. The HLB of a nonionic surfactant is the balance between the hydrophilic and lipophilic portions of the surfactant and is calculated according to the following formula:
HLB=7+11.7×log M_{w}/Mₒ
where M_{w} is the molecular weight of the hydrophilic group portion and Mₒ is the

Examples of silicone surfactants are those sold by Dow Corning under the tradename Dow Corning 3225C Formulation Aid, Dow Corning 190 Surfactant, Dow Corning 193 Surfactant, Dow Corning Q2-5200, and the like are also suitable. In addition, surfactants sold under the tradename Silwet by Union Carbide, and surfactants sold by Troy Corporation under the Troysol tradename, those sold by Taiwan Surfactant Co. under the tradename Ablusoft, those sold by Hoechst under the tradename Arkophob, are also suitable for use in the invention. Such types of silicone surfactants are generally referred to as dimethicone copolyols or alkyl dimethicone copolyols.

Suitable cationic, anionic, zwitterionic, and amphoteric surfactants are disclosed in U.S. Pat. No. 5,534,265, which is hereby incorporated by reference in its entirety.

### Sunscreens

If desired, the compositions of the invention may contain 0.001-20%, preferably 0.01-10%, more preferably 0.05-8% of one or more sunscreens. A sunscreen is defined as an ingredient that absorbs at least 85 percent of the light in the UV range at wavelengths from 290 to 320 nanometers, but transmit UV light at wavelengths longer than 320 nanometers. Sunscreens generally work in one of two ways. Particulate materials, such as zinc oxide or titanium dioxide, as mentioned above, physically block ultraviolet radiation. Chemical sunscreens, on the other hand, operate by chemically reacting upon exposure to UV radiation. Suitable sunscreens that may be included in the compositions of the invention are set forth on page 582 of the CTFA Cosmetic Ingredient Handbook, Second Edition, 1992, as well as U.S. Pat. No. 5,620,965, both of which are hereby incorpated by reference. Examples of such sunscreen materials are p-aminobenzoic acid (PABA), cinoxate, diethanolamine p-methoxycinnamate (DEA-methoxycinnamate), Digalloyl trioleate, dioxybenzone (Benzophenone-8), ethyl 4-[bis-(hydroxypropyl)] amnobenzoate (ethyl dihydroxypropyl PABA), 2-ethylhexyl-2-cyano-3,3-diphenylacrylate (octocrylene), ethylhexyl p-methoxycinnamate (Octyl methoxycinnamate), 2-ethylhexyl salicylate (Octyl salicylate), glyceryl aminobenzoate (Glyceryl PABA), homosalate, lawsone with dihydroxyacetone, menthyl anthranilate, oxybenzone (Benzophenone-3), Padimate A (Pentyl Dimethyl PABA), Padimate 0, (Octyl Dimethyl PABA), 2-Phenylbenzimidazole-5-sulfonic acid (Phenylbenzimidazole Sulfonic acid), Red Petrolatum, Sulisobenzone (Benzophenone-4), triethanolamine salicylate (TEA-Salicylates), and so on.

### Vitamins and Antioxidants

The compositions of the invention may contain vitamins and/or coenzymes, as well as antioxidants. If so, 0.001-10%, preferably 0.01-8%, more preferably 0.05-5% by weight of the total composition are suggested. Suitable vitamins include ascorbic acid and derivatives thereof, the B vitamins such as thiamine, riboflavin, pyridoxin, and so on, as well as coenzymes such as thiamine pyrophoshate, flavin adenin dinucleotide, folic acid, pyridoxal phosphate, tetrahydrofolic acid, and so on. Also Vitamin A and derivatives thereof are suitable. Examples are Vitamin A palmitate, acetate, or other esters thereof, as well as Vitamin A in the form of beta carotene. Also suitable is Vitamin E and derivatives thereof such as Vitamin E acetate, nicotinate, or other esters thereof. In addition, Vitamins D and K are suitable.

Suitable antioxidants are ingredients which assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.

### Humectants

If desired, the compositions of the invention comprise 0.01-30%, preferably 0.5-25%, more preferably 1-20% by weight of the total composition of one or more humectants. Suitable humectants include materials glycols, sugars, and similar materials. Suitable glycols include polyethylene and polypropylene glycols such as PEG 4-240, which are polyethylene glycols having from 4 to 240 repeating ethylene oxide units; as well as C₁₋₆ alkylene glycols such as propylene glycol, butylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on. Preferably, the humectants used in the composition of the invention are C₁₋₆, preferably C₂₋₄ alkylene glycols, most particularly butylene glycol.

### Other Botanical Extracts

It may be desirable to include one or more additional botanical extracts in the compositions. If so, suggested ranges are from about 0.0001 to 10%, preferably about 0.0005 to 8%, more preferably about 0.001 to 5% by weight of the total composition. Suitable botanical extracts include extracts from plants (herbs, roots, flowers, fruits, seeds) such as flowers, fruits, vegetables, and so on, including acacia (dealbata, famesiana, senegal), acer saccharinum (sugar maple), acidopholus, acorus, aesculus, agaricus, agave, agrimonia, algae, aloe, citrus, brassica, cinnamon, orange, apple, blueberry, cranberry, peach, pear, lemon, lime, pea, seaweed, green tea, chamomile, willowbark, mulberry, poppy, and those set forth on pages 1646 through 1660 of the CTFA Cosmetic Ingredient Handbook, Eighth Edition, Volume 2.

### Gellants

It may be desireable to include other gellants in the oil or water phase of the composition to provide gelling. Such gellants may be included a range of about 0.1-20%, preferably about 1-18%, more preferably about 2-10% by weight of the total composition is suggested. Suitable gellants include soaps, i.e. salts of water insoluble fatty acids with various bases. Examples of soaps include the aluminum, calcium, magnesium, potassium, sodium, or zinc salts of C₆₋₃₀, preferably C₁₀₋₂₂ fatty acids.

Also suitable are hydrocolloids such as gellan gum, gum arabic, carrageenan, and those set forth in U.S. Pat. No. 6,197,319 which is hereby incorporated by reference in its entirety.

### Preservatives

The composition may contain 0.001-8%, preferably 0.01-6%, more preferably 0.05-5% by weight of the total composition of preservatives. A variety of preservatives are suitable, including such as benzoic acid, benzyl alcohol, benzylhemiformal, benzylparaben, 5-bromo-5-nitro-1,3-dioxane, 2-bromo-2-nitropropane-1,3-diol, butyl paraben, phenoxyethanol, methyl paraben, propyl paraben, diazolidinyl urea, calcium benzoate, calcium propionate, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, and all of those disclosed on pages 570 to 571 of the CTFA Cosmetic Ingredient Handbook, Second Edition, 1992, which is hereby incorporated by reference.

### Emulsion Stabilizers

If the composition of the invention is in the emulsion form, it may be desirable to incorporate one or more emulsion stabilizers in the composition. If so, suggested ranges are about 0.0001-5%, preferably about 0.0005-3%, more preferably about 0.001-2% by weight of the total composition. Suitable emulsion stabilizers include salts of alkali or alkaline earth metal chlorides or hydroxides, such as sodium chloride, potassium chloride, and the like.

E. Forms of the Cosmetic Composition

The combination of film forming polymers and nonpolar oil may be in the form of a wide variety of cosmetic compositions.

### Foundation Makeup, Color Cosmetics

Foundation makeup or color cosmetics such as eyeshadow, blush, concealer, or eyeliner compositions in the liquid, cream, solid, or stick form. Suitable foundation makeup compositions may be water-in-oil or oil-in-water emulsions. Such compositions generally comprise about:
0.001-80% of a first silicone film forming polymer,
0.001-80% of a second film forming polymer obtained by polymerizing siloxane monomers and ethylenically unsaturated monomers,
0.5-95% water,
0.5-25% particulate matter,
0.01-20% surfactant, and
0.1-95% nonpolar oil.

In addition, these composition may further contain ingredients selected from the group of humectants, preservatives, gellants, and all of the ingredients as set forth above.

Various anhydrous color cosmetic products may also be suitable, such as blush, powder, lipsticks, eyeshadows, and the like. Such anhydrous color cosmetic compositions may generally comprise about:
0.001-80% of a first film forming siloxane polymer,
0.001-80% of a second film forming polymer obtained by polymerizing siloxane monomers and ethylenically unsaturated monomers,
0.1-99% nonpolar oil,
0.1-80% particulate matter; and optionally
0.001-50% wax.

### Lotions, Creams Gels, and Sunscreens

The cosmetic compositions of the invention may be in the form of lotions, gels or sunscreens. Suitable skin care lotions and creams are in the emulsion form, and may be water-in-oil or oil-in-water emulsions, preferably oil-in-water emulsions. Creams, lotions, and/or may contain the following ranges of ingredients:
about 0.001-80% of a first film forming siloxane polymer,
about 0.001-80% of a second film forming polymer obtained by polymerizing siloxane monomers and ethylenically unsaturated monomers,
about 0.1-90% nonpolar oil, and
about 0.01-20% surfactant.

### Skin and Hair Cleansing and Conditioning Compositions

Skin and hair cleansing and conditioning compositions such as facial cleansers, shampoos, hair conditioners and the like are also suitable cosmetic compositions in accordance with the invention.

Generally skin and hair cleansing compositions comprise about:
0.001-80% of a first film forming siloxane polymer,
0.001-80% of a second film forming polymer obtained by polymerizing siloxane monomers and ethylenically unsaturated monomers,
1-95% water, and
0.1-40% surfactant, preferably an anionic, amphoteric, or zwitterionic surfactant.
0.01-40% nonpolar oil.

Suitable hair conditioner compositions comprise:
0.001-80% of a first film forming siloxane polymer,
0.001-80% of a second film forming polymer obtained by polymerizing siloxane monomers and ethylenically unsaturated monomers,
0.1-20% cationic surfactant,
0.1-30% fatty alcohol,
0.001-10% nonionic surfactant, and
5-95% water.

Suitable cationic and nonionic surfactants are as mentioned herein. Examples of suitable fatty alcohols include those having the general formula R—OH, wherein R is a C₆₋₃₀ straight or branched chain, saturated or unsaturated alkyl.

### Nail Enamel Compositions

The cosmetically acceptable carrier for use may also comprise nail enamel compositions. Such compositions generally comprise:
0.001-80% of a first film forming siloxane polymer,
0.001-80% of a second film forming polymer obtained by polymerizing siloxane monomers and ethylenically unsaturated monomers,
0.01-80% solvent,
0.001-40% particulate matter, and
optionally 0.01-40% of one or more polymers such as cellulosic polymers, acrylate polymers, and the like.

Suitable solvents include acetone, alkyl acetates, and the like.

The invention will be further described in connection with the following examples which are set forth for the purposes of illustration only.

### EXAMPLE 1

Lipstick compositions were made as follows:

| Ingredient | 1 | 2 | 3 | 4 | 5 | 6 |
|---|---|---|---|---|---|---|
| Trimethylsiloxy- | 10.00 | 20.00 | 10.00 | 10.00 | 24.40 | 24.40 |
| silicate 803 (MQ | | | | | | |
| resin) | | | | | | |
| Polysilicone 6 | 20.00 | 10.00 | 20.00 | 20.00 | — | — |
| (silicone acrylate | | | | | | |
| copolymer) | | | | | | |
| KP 561 | — | — | — | — | 24.40 | 12.20 |
| Isododecane | 57.00 | 57.00 | 56.00 | 57.00 | 38.20 | 50.40 |
| Polyethylene | 3.00 | 3.00 | — | 3.00 | — | 3.00 |
| 12-hydroxystearic | — | — | 4.00 | — | 3.00 | — |
| acid | | | | | | |
| FD&C Yellow #5 | 1.47 | 1.47 | 1.47 | 1.47 | — | — |
| Aluminum Lake | | | | | | |
| D&C Red #7 | 1.42 | 1.42 | 1.42 | 1.42 | 0.70 | 0.70 |
| Calcium Lake | | | | | | |
| Iron oxide red | 1.57 | 1.57 | 1.57 | 1.57 | 5.00 | 5.00 |
| Black iron oxide | 0.83 | 0.83 | 0.83 | 0.83 | — | — |
| Titanium dioxide | 4.71 | 4.71 | 4.71 | 4.71 | 3.50 | 3.50 |
| Mica | — | — | — | — | 0.80 | 0.80 |

The compositions were prepared by grinding the pigments in a portion of the isododecane. The waxes were melted and the remaining oily ingredients, pigments, and silicone film formers were added and mixed well. The compositions were poured into cosmetic vials and allowed to cool. The resulting compositions were a semi-solid gel like consistency.

### EXAMPLE 2

Lipstick compositions are prepared as follows.

| Ingredient | 1 | 2 | 3 | 4 | 5 | 6 |
|---|---|---|---|---|---|---|
| X-22-8283* | 60.00 | 55.0 | 50.00 | 70.00 | 45.00 | 60.00 |
| Trimethyl- | 4.50 | 7.30 | 8.00 | 2.00 | 20.00 | 4.50 |
| siloxysilicate | | | | | | |
| Isododecane | 16.90 | 18.20 | 20.70 | 11.80 | 8.40 | 17.70 |
| Nonvolatile | 1.50 | 2.20 | 2.00 | 1.00 | 4.20 | 2.00 |
| dimethicone | | | | | | |
| Linear volatile | 1.50 | — | 1.00 | — | — | — |
| dimethicone | | | | | | |
| C12-15 alkyl | — | — | — | — | — | 0.1 |
| benzoate | | | | | | |
| Pentaerythritol | — | — | — | 0.10 | — | — |
| tetraoctanoate | | | | | | |
| Octyl | — | — | — | 0.10 | — | — |
| palmitate | | | | | | |
| Dibutyl | — | — | 0.1 | — | — | — |
| adipate | | | | | | |
| Capric/ | — | — | 0.1 | — | — | — |
| caprylic | | | | | | |
| triglyceride | | | | | | |
| Dioctyl malate | 0.10 | 0.1 | — | — | — | — |
| Octyl | — | 0.1 | — | — | — | — |
| isononanate | | | | | | |
| Trioctyl- | — | 0.1 | — | — | — | — |
| dodecyl citrate | | | | | | |
| Neopentyl | — | — | 0.1 | — | — | — |
| glycol | | | | | | |
| dioctanoate | | | | | | |
| Quaternium- | 5.00 | 5.00 | 8.00 | 5.00 | 5.00 | 5.00 |
| 18 hectorite/ | | | | | | |
| isododecane/ | | | | | | |
| propylene | | | | | | |
| carbonate | | | | | | |
| Organic and | 6.00 | 8.00 | — | 6.00 | — | 6.00 |
| inorganic | | | | | | |
| pigments | | | | | | |
| Mica/Pearl | 2.00 | 2.00 | — | 2.00 | 1.00 | 2.00 |
| Pigments/ | — | — | 8.00 | — | — | — |
| mica/ | | | | | | |
| pearl | | | | | | |
| 40% pigments | — | — | — | — | 15.00 | — |
| in | | | | | | |
| isododecane | | | | | | |
| C20-40 | 2.00 | — | — | — | 1.40 | — |
| alcohol | | | | | | |
| Oleyl alcohol | — | — | — | — | — | 0.10 |
| Isostearyl | — | — | — | — | — | 0.10 |
| alcohol | | | | | | |
| Synthetic | — | 2.00 | — | 2.00 | — | — |
| wax | | | | | | |
| Polyethylene | — | — | 2.00 | — | — | 2.00 |
| Methyl | 0.30 | — | — | — | — | 0.30 |
| paraben | | | | | | |
| Propyl | 0.10 | — | — | — | — | 0.10 |
| paraben | | | | | | |
| BHT | 0.10 | — | — | — | — | 0.10 |

*X-22-8238 from Shin-Etsu, acrylates dimethicone copolymer, 40% in isododecane

The compositions are prepared by grinding the pigments in a portion of the isododecane. The waxes are melted and the remaining oily ingredients, pigments, and silicone film formers are added and mixed well. The compositions are poured into cosmetic vials and allowed to cool.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

1. A cosmetic composition comprising a first film forming siloxane polymer, and a second film forming polymer obtained by polymerizing siloxane monomers and at least one monomer selected from the group consisting of ethylenically unsaturated monomers, urethanes, amides, and mixtures thereof, said polymers solvated or dispersed in a cosmetically acceptable nonpolar oil.

2. The composition of claim 1 wherein the first film forming siloxane polymer is a silicone resin.

3. The composition of claim 3 wherein the silicone resin is a T resin, an MT resin, and MQ resin or mixtures thereof.

4. The composition of claim 3 wherein the silicone resin is a T resin.

5. The composition of claim 4 wherein the T resin comprises alkoxy and/or hydroxy groups.

6. The composition of claim 2 wherein the silicone resin is an MT resin.

7. The composition of claim 6 wherein the MT resin is of the general formula MₓT_{y} wherein M is R₁R₂R₃SiO_{1/2}; T is RSiO_{3/2}.

8. The composition of claim 7 wherein the MT resin additionally comprises one or more difunctional units.

9. The composition of claim 3 wherein the MQ resin is of the general formula MₓQ_{y} wherein M is R₁R₂R₃SiO_{1/2}; Q is SiO_{4/2}; R₁, R₂, and R₃ are each independently C₁₋₃₀ straight or branched chain alkyl or phenyl; and x and y are each independently 1-1,000,000.

10. The composition of claim 9 wherein R₁, R₂, and R₃ are each independently methyl or phenyl.

11. The composition of claim 10 wherein the MQ resin has alkoxy or hydroxy functional groups.

12. The composition of claim 1 wherein the second film forming polymer is obtained by polymerizing one or more M, D, T, or Q units with one or more ethylenically unsaturated monomers, or an amide or urethane.

13. The composition of claim 12 wherein the second film forming polymer is obtained by polymerizing one or more M, D, T, or Q units with one or more ethylenically unsaturated monomers.

14. The composition of claim 13 wherein the ethylenically unsaturated monomer is of the general formula: 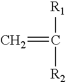
wherein R₁, and R₂ are each independently H, halogen, hydroxyl, fluoroalkyl, a C₁₋₃₀ straight or branched chain alkyl, aryl, aralkyl; R₂ is a pyrrolidone, or a substituted or unsubstituted aromatic, alicyclic, or bicyclic ring where the substitutents are C₁₋₃₀ straight or branched chain alkyl, or COOM or OCOM herein M is a C₁₋₃₀ straight or branched chain alkyl, pyrrolidone, or a substituted or unsubstituted aromatic, alicylic, or bicyclic ring where the substitutents are C₁₋₃₀ straight or branched chain alkyl.

15. The composition of claim 14 wherein the ethylenically unsaturated monomer is an acrylate or methacrylate.

16. The composition of claim 15 wherein the second film forming polymer is a silicone acrylate copolymer.

17. The composition of claim 1 wherein the nonpolar oil is a paraffinic hydrocarbon.

18. The composition of claim 17 wherein the paraffinic hydrocarbon is volatile.

19. The composition of claim 1 which is an anhydrous pigmented composition.

20. The composition of claim 1 which is a lipstick
